(19) United States
(12) Patent Application Publication
(10) Pub. No.: US 2007/0270376 A1
(43) Pub. Date: Nov. 22, 2007

(54) Combinations of Hyaluronic Acid and Polyunsaturated Fatty Acids

(75) Inventor: Anthony Michael Chandler, Surrey (GB)

Correspondence Address: CONLEY ROSE, P.C.;David A. Rose, P. O. BOX 3267, HOUSTON, TX, 77253-3267, US

(73) Assignee: Bionovate Limited, Cambridgeshire (GB)

(21) Appl. No.: 11/569,207

(22) PCT Filed: May 17, 2005
(86) PCT No.: PCT/GB05/01890

§ 371 Date: Apr. 20, 2007

(30) Foreign Application Priority Data
May 19, 2004 (GB) 0411165.4

(51) Int. Cl. A61K 31/726; A61P 19/02

(52) U.S. Cl. 514/54

(57) ABSTRACT

A pharmaceutical or veterinary composition comprises a hyaluronic acid or a salt thereof or an ester of hyaluronic acid with an alcohol of the aliphatic, heterocyclic or cycloaliphatic series, or a sulphated form of hyaluronic acid, together with at least one eicosanoid or tetraenoic polyunsaturated fatty acid or an ester or a salt thereof, preferably in the form of an extract of fatty acids from the New Zealand Green Lipped Mussel Perna canaliculus. The compositions are active against inflammatory conditions including osteoarthritis.

This invention relates to the synergistic effect of combined omega-3 series eicosanoid polyunsaturated fatty acids and hyaluronic acid upon inflammatory conditions, including rheumatoid and osteoarthritis.

Mammalian inflammatory pathways are an important consequence of the immune system and play a vital role in the normal homeostasis of the body. Whilst short-term inflammation has a protective function, in chronic diseases such as arthritis, inflammation is associated with the typical oedema, swelling and pain.

Arthritis is a major chronic disease worldwide that produces an enormous socioeconomic burden. Arthritis continues to be of considerable impact to the lives of millions and is believed to affect 15% of the population in its chronic form. The disease is simply described as inflammation of joints due to physical degeneration of the joint structure. The commonest form is degenerative joint disease (DJD) or osteoarthritis which involves the physical degeneration of cartilage exposing sub-chondral bone, thereby inducing an inflammatory response.

The use of Polyunsaturated Fatty Acids (PUFAs) such as the omega-3 and omega-6 series in the amelioration of inflammation in arthritis has been well documented. PUFAs influence the mammalian inflammatory pathways due to their interaction with the metabolism and supply of arachidonic acid into the cyclo-oxygenase and Lipoxygenase enzyme pathways that produce potent prostaglandins and leukotrienes respectively.

Prostaglandins and leukotrienes are potent biologically active structures that normally play an essential role in tissue homeostasis. However, following cellular injury or trauma the respective production of specific prostaglandins and leukotrienes shifts to an inflammatory reaction with local physiological effects [see Table 1].

What is perhaps to some extent less widely appreciated is the structural similarities exhibited by these essential physiological mediators and in particular their shared metabolic precursor, arachidonic acid. Arachidonic acid, prostaglandins and leukotrienes are PUFA structures with a 20-carbon chain and are therefore described as Eicosanoids. They are synthesised in almost every tissue but are not stored in any significant quantities. These eicosanoid PUFAs therefore act as the precursor to the arachidonic acid cascade.

TABLE 1
Source and physiological response produced by some
of the products of the arachidonic acid cascade.

| Eicosanoid | Primary source | Physiologic response |
|---|---|---|
| Prostaglandin D₂ | Mast cell, | Vasodilation, bronchoconstriction |
| (PGD₂) | multiple other | |
| | tissues | |
| Prostaglandin | Multiple | Vasoconstriction, uterine and |
| F₂ₐₗₚₕₐ (PGF₂ₐₗₚₕₐ₎ | tissues | bronchial smooth muscle contraction |
| Prostacyclin | Vascular | Vasodilation, inhibits platelet |
| (PGI₂) | endothelium, | aggregation, acute inflammatory |
| | macrophages | reactions |
| Thromboxane A₂ | Platelets, | Vasoconstriction, platelet |
| (TXA₂) | white blood | aggregation |
| | cells | |
| Prostaglandin E₂ | White blood | Vasodilation, acute inflammatory |
| (PGE₂) | cells, | response, inhibits gastric acid |
| | multiple other | secretion, pyrexia, analgesia, |
| | tissues | inhibits renal tubular |
| | | reabsorption, stimulates |
| | | osteoclastic activity |

Eicosanoid Metabolism

Eicosanoids are 20-carbon compounds derived from polyunsaturated fatty acids, also known as the eicosanoic acids and which serve as precursors to a variety of other biologically active compounds within cells. These include prostaglandins, thromboxanes and leukotrienes, which are themselves eicosanoids and are therefore based upon the eicosanoid 20-carbon structure.

At the cellular level, arachidonic acid is one of the major sources of 20-carbon structures which provide the essential precursors of prostaglandins (sometimes referred to as prostanoids), thromboxanes and leukotrienes. These compounds act as biological regulators within animals and their function depends upon the type of tissue and relevant enzyme systems involved and are well known mediators of inflammation and immune response.

Eicosanoid metabolism is controlled by the availability of arachidonic acid or other eicosanoid structures, enzyme expression and negative or positive feedback loops for example. Eicosanoids are potent regulators of cell metabolism but have a short half-life of less than 5 minutes allowing for significant control over physiological functions. Their potency is such that the ratio of body mass to eicosanoid mass is in the order of 1 million.

In recent years pharmacological research has begun to unravel the complexities of mammalian inflammatory pathways leading to increased pharmaceutical interest in novel compounds that can provide anti-inflammatory activity with reduced adverse effects, contra-indications or toxicity.

In the following description of the invention and the background to it, reference will be made to the figures of the drawings appended hereto which show:

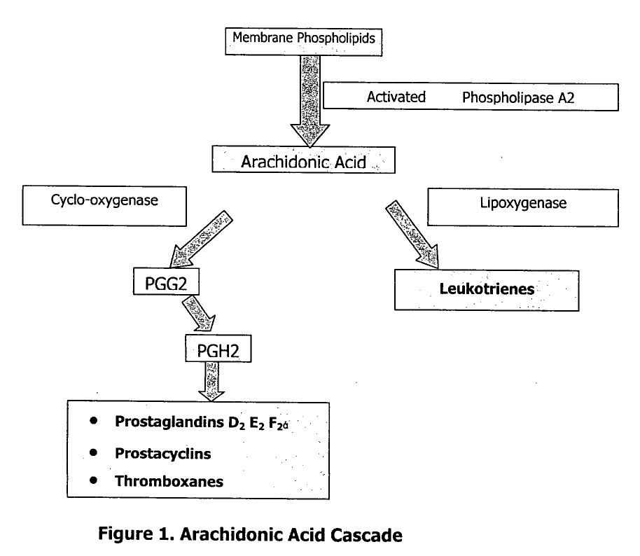

FIG. 1: shows an illustration of the Arachidonic Acid Cascade;

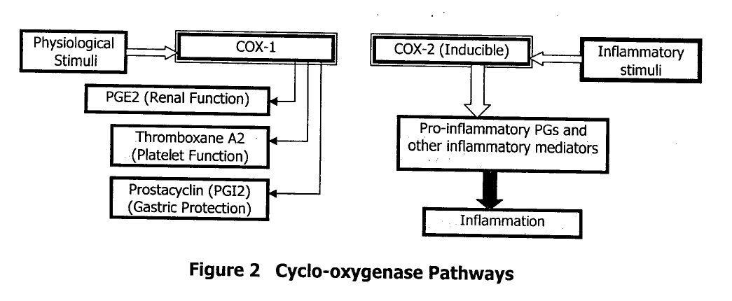

FIG. 2: shows an illustration of the two cyclo-oxygenase pathways;

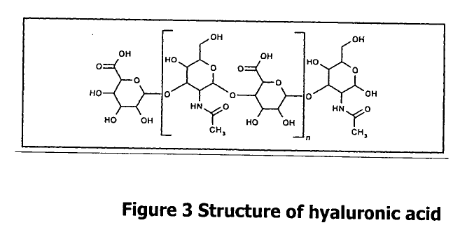

FIG. 3: shows the structure of hyaluronic acid; and

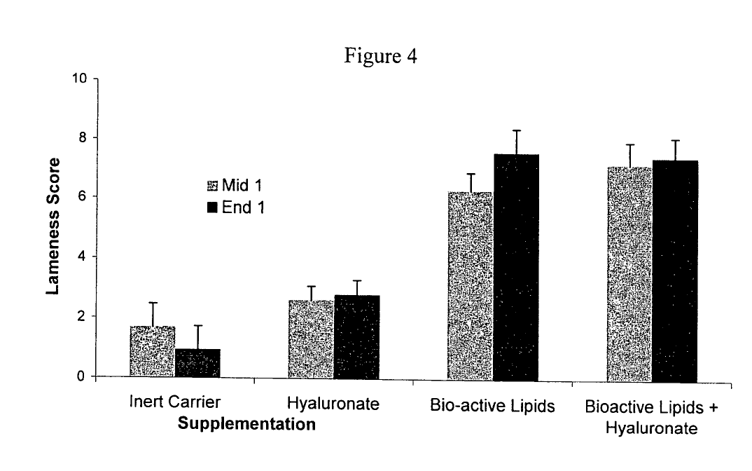

FIG. 4: shows results obtained in the Example.

Eicosanoids and the Inflammatory Process

The inflammatory process begins with cell injury. Trauma, infection, or other injury to the cell which activates membrane bound phospholipase A2 (pLA2), which releases arachidonic acid from the injured cell's membrane. Arachidonic acid fuels the cyclo-oxygenase and lipoxygenase inflammatory pathways.

The inflammatory process directly involves eicosanoid metabolism. Of the numerous mechanisms involved a number of pathways are of particular interest, the cyclo-oxygenase (or COX) and lipoxygenase (LOX) pathways, both of which constitute the Arachidonic Acid Cascade shown in FIG. 1.

The arachidonic acid cascade is responsible for the production of various biological regulators at the tissue level. Control of eicosanoid metabolism can be achieved by the supply of arachidonic acid, negative feedback mechanisms and therapeutically by treatment with non-steroidal anti-inflammatory drugs (NSAIDs) for example.

The biochemical by-products of this process have been implicated in many divergent physiologic responses to inflammation: vasodilation, bronchoconstriction, vasoconstriction, smooth muscle contraction, platelet aggregation, pyrexia, analgesia, inhibition of renal tubular sodium re-absorption, stimulation of osteoclastic activity and inhibition of gastric acid secretion (see Table 1).

The Lipoxygenase Pathway

Lipoxygenase is an enzyme that converts arachidonic acid to several intermediates, including 5-hydroperoxyeicosatetraenoic acid (5-HPETE), which gives rise to the leukotrienes (LTA₄, LTB₄, LTC₄, and LTD₄). Leukotrienes play a role in vascular permeability and they are potent chemotactic factors, increasing White Blood Cell (WBC) migration into inflamed tissues. Leukotrienes are associated with the development of oedema and WBC effusion into tissues such as joints in arthritis patients.

In arthritis most research has concentrated on treatment with non-steroidal anti-inflammatories (NSAIDs). The widely varying profiles of currently available NSAIDs may be explained by the discovery of two isoforms of the cyclo-oxygenase enzyme possessing different profiles, see FIG. 2.

Cyclo-oxygenase 1 (COX 1) has a physiological role and influences the normal activities of platelet aggregation, gastric mucosa, and kidney. COX1 activity is not influenced by inflammatory stimulation.

Cyclo-oxygenase 2 (COX 2) is induced by inflammatory stimulation releasing pro-inflammatory prostaglandins.

The increased production of prostaglandins accompanying the arachidonic acid cascade is regulated by the supply of arachidonic acid. The inflammatory reaction is therefore a two stage process; increased enzyme expression, and increased arachidonic acid supply.

Thus it follows that the inflammatory reaction is dependent upon the availability of supply of arachidonic acid. It also follows that the inflammatory process can be influenced by the manipulation of the arachidonic acid concentration and therefore is dependent upon the availability of PUFAs.

Arachidonic acid production and availability at the cell membrane depends upon dietary intake of essential fatty acids such as omega-6 linoleic acid. Its release from the cell membrane by phospholipase A2 clearly can influence the availability of this vital eicosanoid precursor at the active site of COX and LOX enzymes.

Naturally Occurring Eicosanoids and the Role of PUFAs

The most recognised naturally occurring Eicosanoids are found in marine-derived oils such as fish oils which contain the omega-3 series of Polyunsaturated Fatty Acids (PUFAs). Fish oil is a well known source of one such eicosanoid in particular, namely eicosapentaenoic acid or EPA. EPA has been used for many years with little if any evidence of clinical anti-inflammatory activity at the dose commonly used.

PUFAs are not only required for energy, but are implicated in the regulation of biochemical pathways within the body. In particular, PUFAs are the obligate precursors of a wide range of signalling molecules, including the prostanoids, which have a central role in inflammatory responses. Thus altering dietary PUFA composition may have a considerable influence on the inflammatory response through alterations in the type and relative quantities of prostanoids synthesised.

In general, the 2-series prostaglandins (derived from n-6 PUFAs) are far more pro-inflammatory than the 3-series prostaglandins (derived from n-3 PUFAs), so increases in the proportion of n-3 PUFA precursors in the body should have significant anti-inflammatory effects. The benefits of this are far-reaching as a means for minimising respiratory disease and arthritis concomitant with reduced need for drug intervention.

Further results have shown that n-3 PUFAs inhibit the conversion of the precursor lipid, arachidonic acid by the lipoxygenase and cyclo-oxygenase pathways, to proinflammatory metabolites such as leukotriene B4 (LTB₄), 5-hydroxy-eicosopentaenoic acid (HETE), and thromboxane A2. The leukotrienes. LTC₄, LTD₄ and LTE₄ have been shown to produce strong bronchospastic responses in central and peripheral airways, and reduce airflow dramatically in asthma, adult respiratory distress syndrome, hypoxic pulmonary hypertension and LPS-induced pulmonary injury. The n-3-PUFA linolenic acid has been shown to reduce leukotriene production in adult asthmatics.

It has been demonstrated in knock-out mice, that a deficiency of PGHS-1 and PGHS-2 (the key prostaglandin synthetic enzymes), greatly reduces the inflammatory response in allergic lung responses. These studies confirm the importance of arachidonic acid metabolites in responses to respiratory challenges. Whilst a certain level of eicosanoids is required for ‘housekeeping’ purposes and the establishment of an immune response is a necessary function, the exact quantities and type of prostanoid synthesised may be crucially altered by an imbalance of n-3/n-6 PUFAs resulting in physiological systems such as the pulmonary airways and joints becoming hyper-sensitive to harmful environments and infection. The advantages of using n-3 PUFAs to inhibit arachidonic acid metabolism is that, unlike most commonly used anti-inflammatory drugs, they do not completely block cyclo-oxygenase activity, thus allowing for synthesis of beneficial prostanoids such as prostacyclin and PGE₂.

Pharmacological Application of Lipid-Derived Omega-3 Series Poly-Unsaturated Fatty Acids from Perna Canaliculus

The anti-arthritic properties of the New Zealand Green Lipped Mussel (Perna canaliculus) have been reviewed for nearly 30 years. More recently the range of omega-3 series PUFAs naturally present in Perna canaliculus have been evaluated for their anti-inflammatory and anti-asthmatic properties. These marine-derived lipids have been shown to possess potent anti-inflammatory properties by inhibiting the action of the two enzymes, cyclo-oxygenase and lipoxygenase.

U.S. Pat. No. 63,462,278 describes a method of anti-inflammatory treatment of a human or animal patient comprising administration of a lipid extract of Perna canaliculus. U.S. Pat. No. 6,596,303 describes the alleviation of arthritic symptoms in animals by administering powdered Perna canaliculus in the feed. WO03043570A2 describes formulations and methods of treatment of inflammatory conditions comprising an omega-3 fatty acid, such as DHA, or a flavonoid with a non-alpha tocopherol. WO03011873A2 describes a phospholipid extract from a marine biomass comprising a variety of phospholipids, fatty acid, metals and a novel flavonoid. WO02092450A1 describes the production and use of polar-rich fractions containing EPA, DHA, AA, ETA and DPA from marine organisms and others and their use in humans food, animal feed, pharmaceutical and cosmetic applications.

The lipids extracted from the Green Lipped Mussel have been shown to contain particular types of fatty acids not found in the same proportion in other organisms. These omega-3 series PUFAs have only recently been characterized due to advances in manufacturing. It is essential that cold processing and suitable drying methods are used to preserve the delicate structures of these particular fatty acids. The omega-3 series content is known to include the PUFAs: EPA, DHA and the ETAs (eicosatetraenoic acids).

The ETAs have a similar structure to the omega-6 series arachidonic acid but have been shown to be profoundly more potent than EPA, DHA or a-LNA in inhibiting the production of proinflammatory prostaglandins, thromboxanes and leukotrienes. ETAs have been shown to be as potent as ibuprofen and aspirin in independent studies and 200 times more potent than EPA in the rat paw oedema test (Whitehouse M W et al, Inflammopharmacology 1997;5:237-246).

Pharmacologically, lipid derived from Perna canaliculus has been shown to significantly inhibit cyclo-oxygenase 2 and Lipoxygenase pathways following in vitro studies that determined the IC₅₀ for each:
Cyclo-oxygenase 2 IC₅₀=1.2 μg/ml
Lipoxygenase IC₅₀=20 to 50 ug/ml
   Therefore, the lipids occurring naturally in Perna canaliculus exhibit significant anti-inflammatory activity in vitro and in vivo.
   Hyaluronic Acid and the Treatment of Arthritis

Hyaluronic acid (HA) is a high molecular weight glycosaminoglycan, or GAG, which plays a vital role in the functioning of extracellular matrices. HA is also important in that it has numerous actions in the mechanisms associated with inflammation and the wound healing process.

HA is a polymer of glucuronic acid and N-acetylglycosamine, bonded alternatively by glycosidic beta (1,3) and beta (1,4) bonds (FIG. 3). Hyaluronic acid interacts with other proteoglycans and collagen to give stability and elasticity to the extracellular matrix of connective tissue and has essential physico-chemical properties vital to healthy periodontal tissue.

Hyaluronic acid binds to different proteins and water molecules by means of hydrogen bonds to form a viscous macroaggregate whose primary function is to regulate the hydration of tissues, the passage of substances in the interstitial compartment and the structure of connective tissue extracellular matrix. Hyaluronic acid is highly viscous and is found in a wide variety of body tissues e.g. vitreous humour of the eye, synovial fluid, umbilical cord, cartilaginous tissue, synovium, the skin, the mucosa of the oral cavity. The polymer can bind up to 50 times its own weight of water and associates with specific proteins and tissue components. HA forms a viscous cement, regulates the water content of the tissue, controls the movement of substances (nutrients, toxins etc.) into the extra-cellular spaces and prevents the formation of oedemas which occur on tissue inflammation or injury.

In addition, hyaluronic acid binds to cellular receptors that are expressed only in cells in active division, it also acts as a regulator of migration and cellular division mechanisms which are especially important in healing and tissue repair.

Normal joint structure consists of two adjoining bones capped with cartilage and sealed by the synovial membrane, which itself encloses synovial fluid that acts as a cushion to dampen the compressive forces occurring when the joint is compressed. Synovial fluid also has various physiological functions providing for a healthy cartilage and synovial membrane.

Cartilage is a form of specialised connective tissue designed to be tough and flexible. It is composed of extracellular matrix with embedded protein collagenous structures to give it tensile strength but retaining a smooth physical surface.

The extracellular matrix is a complex structure consisting of various polymers of amino sugars and sugar molecules in long glycosaminoglycan chains binding to proteins to form a mesh of supportive structures; the proteoglycans.

GAGs also include glucosamine and chondroitin. The link between proteo-glycans and collagens that underlie the structure of cartilage is hyaluronic acid.

Without HA the cartilage structure breaks down and this is typically seen when subchondral bones are exposed in arthritis producing catabolic enzymes that hydrolyse HA to shorter chain lengths. As the extracellular cement unravels its structure more GAGs are lost and hydrolysed. Indeed there is an inverse correlation between the severity of arthritis and loss of GAGs in a joint.

Clinically, there are three requirements for the management of arthritis:

1. Control inflammation and therefore pain

2. Maintain mobility

3. Reduce joint degeneration, or its progress.

HA is the most important GAG present in connective tissue, such as joint cartilage. It is required to form 50% of the synovial fluid as well as linking protein to proteoglycans, so acting as the “backbone” of connective tissue structure.

Historically, HA has been administered by orthopaedic surgeons as intra-articular injection directly into the joint for the treatment of arthritis and has clinical uses in veterinary as well as human medicine. It is also used in ophthalmology, burn dressings and dermatology, particularly wound healing, implant technology and surgery to prevent adhesions.

U.S. Pat. No. 6,607,745 describes oral administration of hyaluronic acid with a food acceptable carrier, which may be food or water, at a dosage of 0.1 μg to 400 μg/kg of body weight as an anti-inflammatory.

A commercial feed supplement for horses marketed as Hylaron comprises hyaluronic acid and flax seed, contributing omega-3 and omega-6 fatty acids. However, flax seeds are not a good source of eicosanoid fatty acids, they are instead rich in linolenic acid. This is not equivalent in its biological effects to the long-chain omega-3 fats found in marine oils. The eicosanoids are more rapidly incorporated into plasma and membrane lipids and produce more rapid effects than does linolenic acid. Experimental studies suggest that intake of 3-4 grams of linolenic acid per day is equivalent to 0.3 grams eicosanoids per day. Am. J. Clinical Nutrition, September 1999; 70: 560-569.

Percutaneous Transport and Absorption

Percutaneous absorption of chemicals for therapeutic benefit has always been the basis for topical treatments in dermatology. More recently, the use of this method of administration has gained additional interest with the development of transdermal technology to provide an alternative to traditional intravenous (iv) or oral routes of administration.

Percutaneous absorption has a number of applications not the least being to treat the exterior skin, underlying structures (e.g. structures surrounding a joint) or to provide alternative routes to achieve systemic concentrations of target compounds.

The healthy skin is an impermeable barrier to the loss of hydration from within the body and invasion of foreign material from external sources. Developing treatments for external application must reflect the desired functional rationale for the treatment (i.e. skin surface application, underlying structures or systemic targets). Each requires different functional components to help permeate the relevant structures in the skin.

Percutaneous absorption refers to the absorption of topical medications through the epidermal barrier into underlying tissues and structures with transfer into the systemic circulation. The outermost layer of the epidermis, the stratum cornea, forms the important barrier that regulates the amount and rate of percutaneous absorption.

The formation of this barrier is accomplished through the intercellular lipids along with corneocytes; the primary cell of the epidermis. The lipids comprise free fatty acids, ceramides, as well as cholesterol and are deposited in the intercellular spaces within the stratum corneum. The intercellular lipids provide the primary barrier to molecular movement across the stratum corneum by allowing diffusion at a rate 1,000-fold less than is allowed by cellular membrane.

Corneocytes are cells that have differentiated into structures that contain primarily proteins and only 15% to 30% water. In comparison, other living cells contain approximately 80% to 90% water. The dry corneocytes and hydrophobic intercellular lipids comprise a highly organized and differentiated structure that forms an effective barrier to passage of substances to underlying tissues.

Percutaneous absorption of topically applied medications is accomplished by the process of passive diffusion. It requires substances to pass through the stratum corneum and epidermis, diffuse into the dermis, and eventually transfer into the systemic circulation. Diffusion occurs down a concentration gradient resulting in the dilution of compounds as they progress along the gradient. In addition, the compound may be bound or metabolised as it passes through the underlying tissues. All of these factors will affect the potency of the medication, the level of systemic absorption, and ultimately its efficacy.

Topically applied medication therefore must be developed with the correct components to provide adequate penetration for the required use. Most topically applied substances, particularly nonpolar or hydrophobic compounds, are absorbed by diffusion across the stratum corneum and epidermis through the intercellular corridors. However, polar or hydrophilic substances are transported through the transcellular absorption route. Hair follicles and eccrine sweat ducts may also serve as diffusion shunts for certain substances such as ions, polar compounds, and very large molecules that would otherwise move through the stratum corneum very slowly because of their high molecular weight.

Skin characteristics are an essential consideration for percutaneous absorption. Features of normal skin, barrier changes in the skin, and vascular changes in the skin all play a critical role in absorption. One of the most important factors affecting percutaneous absorption is skin hydration and environmental humidity. In the normal state of skin hydration, the stratum corneum may be penetrated only by medications passing through the tight, relatively dry, lipid barrier between cells. However, when the skin is hydrated, water molecules bind to hydrophilic lipids between the corneocytes and enable water-soluble medications to more easily diffuse. Therefore, absorption of topical therapies is enhanced by hydration of the skin.

Several additional characteristics of the skin can affect percutaneous absorption of an applied medication. Increased cutaneous vasculature or vasodilatation at the site of application which frequently occurs with inflammation can enhance both local and systemic effects of the drug. This, along with increased surface area of the drug application, will boost overall percutaneous absorption.

The rate-limiting factor of percutaneous absorption seems to be diffusion through the stratum corneum and hence the effectiveness of the epidermal permeability barrier correlates inversely with percutaneous absorption.

Therefore, to increase the efficiency of diffusion into and beyond the stratum corneum, a penetration enhancer can be included in the formulation of the topically applied medication. This material increases the rate of diffusion into the tissues so enhancing the therapeutic effect by increasing the percutaneous concentration of active material, or achieving the same rate of diffusion with a lower initial concentration of topically applied material.

Delivery is an important issue in the development of any drug product, and the choice of a delivery route is contingent upon optimising drug delivery while maintaining convenience and ease of administration.

Transdermal drug delivery provides excellent control of the rate of delivery directly into the bloodstream. It also offers a predictable pharmacokinetic profile and constant drug levels over extended periods of time without the extreme peak/trough fluctuations inherent in oral administration.

Transdermal patches offer benefits similar to those of oral administration in that both are easy for patients to self-administer and place few restrictions on patients daily activities. Transdermal drug delivery offers the best of IV and oral administration

### SUMMARY OF THE INVENTION

The invention provides a pharmaceutical or veterinary composition comprising a hyaluronic acid or a salt thereof or an ester of hyaluronic acid with an alcohol of the aliphatic, heterocyclic or cycloaliphatic series, or a sulphated form of hyaluronic acid, together with at least one eicosanoid or tetraenoic polyunsaturated fatty acid an ester or a salt thereof. The eicosanoid or tetraenoic fatty acid may be present as free fatty acid, or as a triglyceride, diglyceride or other ester, e.g. a methyl or ethyl ester. Eicosanoid glycerides may be mixed glycerides in which a non-eicosanoid fatty acid is present also.

In a first preferred practice of the invention the composition is for topical administration. Suitably, it comprises a pharmaceutically or veterinarily acceptable diluent or carrier. Such a diluent may be water, preferably sterile water, or may be organic solvent, or vegetable oil-based. It may contain skin penetrant ingredients serving to speed penetration of the skin by the active ingredients. These include for instance menthol or non-ionic surfactants or ionic surfactants or mixtures of these. The compositions may comprise stabilising ingredients such as anti-oxidants, suitable anti-oxidants include vitamin C (ascorbic acid), or vitamin E (alpha tocopherol). The composition may also include salts to buffer the solution to physiological pH.

Topical formulations may be formulated as a cream, ointment, lotion, poultice or gel, or they may be incorporated into a patch to be applied to the skin, the patch may have a single or multilayer construction.

Preferred topical compositions may contain a concentration of hyaluronic acid or a said derivative thereof in an amount of from 1 to 20% (w/w) or from 5 to 15% (w/w) or from 10 to 20% (w/w) based on the total weight of the composition. The compositions preferably contain a concentration of said eicosanoid or tetraenoic fatty acid or derivative thereof in an amount of 1 to 20% (w/w) or from 5 to 15% (w/w) or from 10 to 20% (w/w) based on the total weight of the composition.

In an alternative preferred aspect, compositions of the invention are for oral administration. Such compositions may again comprise a pharmaceutically or veterinarily acceptable diluent or carrier. Suitable examples of carriers include water, preferably sterile, or a vegetable oil. Such compositions may be formulated as a syrup, solution, capsule, lozenge, tablet, chewable tablet, rapid dissolving wafer, or gelatin or non-gelatin capsule. The actives may be absorbed onto a powder carrier such as lactose and formed into a conventional tablet. For rectal administration a suppository format may be used.

The composition may be in unit dosage form, wherein each unit dosage form contains from 5 to 500 mg or from 10 to 250 mg or from 20 to 50 mg of hyaluronic acid or said derivative thereof. Such a composition in unit dosage form may be such that each unit dosage form contains from 5 to 500 mg or from 10 to 250 mg or from 20 to 50 mg of said eicosanoid or tetraenoic fatty acid or derivative thereof.

Liquid dosage forms may be put up in unit dose format, e.g. in sachets of a single dose or may be presented in multiple dose format, e.g. in a bottle containing several or many doses. Compositions in liquid dosage form may suitably contain a concentration of from 1 to 20% (w/v) of hyaluronic acid or said derivative thereof or from 5 to 15% (v/v) or from 10 to 15% (v/v). They may contain a concentration of from 1 to 20% (w/v) of said eicosanoid or tetraenoic fatty acid or said derivative thereof or from 5 to 15% (v/v) or from 10 to 15% (v/v).

Oral formulations of the invention may be presented as food or feed supplements or for addition to drinking water.

In all of these compositions, the weight ratio of said hyaluronic acid or derivative thereof to said eicosanoid or tetraenoic fatty acid or derivative thereof is from 1 to 1, 1 to 5, 1 to 10, up to 1 to 100.

For the reasons explained above, said eicosanoid or tetraenoic fatty acid or derivative thereof is preferably provided as an extract of fatty acids from Perna canaliculus. This may be an unselected extract of fatty acids from Perna canaliculus or may be especially enriched in eicosanoid or tetraenoic fatty acids either through purification from a starting extract or by the choice of extraction conditions being such as to favour the extraction of the eicosanoid or tetraenoic fatty acids with respect to non-eicosanoid fatty acids. In particular, it is preferred that the eicosanoid fatty acid is or comprises eicosatetraenoic acid. In particular, it is preferred that the eicosanoid fatty acid is or comprises ω-3 eicosatetraenoic acid and preferably constitutes at least 0.05 (w/w) of the fatty acid content of the composition. Or from 0.05 to 3% (w/w) or from 0.1 to 1.0% (w/w).

A number of forms of hyaluronic acids are available from various sources. These include natural sources such as cockerel combs or other animal connective tissue sources and also from bacterial sources such as Streptococcus zoepidicus. The molecular weights of hyaluronic acids range from 50,000 upwards to about 8×10⁶ Daltons. We prefer that said hyaluronic acid or derivative thereof is a low molecular weight form, having a molecular weight of from 50,000 to 500,000, more preferably, having a molecular weight of from 150,000 to 250,000, e.g. about 200,000.

As indicated above topical compositions of the invention may comprise a skin penetration agent such as menthol.

Topical preparations of PUFAs by their physical nature and characteristics will permeate the lipid-rich intercellular area of the stratum corneum. However, this has been found to be chain-length dependent (Drug Development and Industrial Pharmacy (1999), 25(11), 1209-1213)

Therefore the addition of menthol in concentrations of 0.1 to 20 wt %, more preferably 0.1% to 10% (e.g. 1 to 5%) in a suitable carrier to a mixture of polyunsaturated fatty acids, either omega-3 or omega-6 series, will enhance the percutaneous flux of PUFAs into the subcutaneous tissues and systemic circulation. Additionally, other compounds in the topical applications will have improved flux when incorporated into a system containing menthol.

Thus the inclusion of a skin penetration agent is useful in composition for percutaneous application to the skin to treat conditions such as localised inflammation and swelling associated with arthritis of the knees, elbows, shoulders etc or any joint. Compositions may be presented as a cream, lotion or gel to allow percutaneous absorption of the components to the underlying structures such as synovial membranes and capsular tissues.

Transdermal application is an alternative delivery method to oral application for any of the presentations above and specifically for application in arthritics to achieve systemic concentrations sufficient to achieve therapeutic effect. The compositions may be presented as a single or multi-layered system of therapeutic components and menthol as a percutaneous enhancer or as reservoir-based systems where the mixture with menthol is held in a reservoir and released over time through permeable membranes onto the skin. Alternatively, an adhesive-based system can be used where the components, with menthol, are added to the adhesive layer where they permeate the skin.

The invention includes a method of therapy comprising administering to a mammal suffering from an arthritic condition or other inflammatory condition or in need of prophylaxis in respect of such a condition, an effective amount of a hyaluronic acid or a salt thereof or an ester of hyaluronic acid with an alcohol of the aliphatic, heterocyclic or cycloaliphatic series, or a sulphated form of hyaluronic acid, together with at least one eicosanoid or tetraenoic polyunsaturated fatty acid or ester thereof or a salt of a said fatty acid, separately or as an admixture. Glyceride, methyl or ethyl esters may be used. The administration can of course be of a composition according to the invention. Suitable dosages of hyaluronic acid or a derivative thereof will typically be from 0.1 to 100 mg/kg body weight per day or from 1 to 10 mg/kg body weight per day and suitable dosage amounts for the ω-3 eicosanoid or tetraenoic fatty acid component are from 1 to 500 mg/kg body weight per day or from 2 to 100 mg/kg body weight per day.

The incorporation of HA with lipids derived from Perna canaliculus into a formulation for the treatment of arthritis provides the anti-inflammatory activity required with the joint-structure stabilising action of HA. However, a strong and unexpected synergism is obtained between the actions of these therapeutic components.

Lipids from Perna canaliculus demonstrate significant anti-inflammatory activity in vitro and in vivo and have been shown to reduce inflammation in arthritics. However, the lipid extract has no long-term effect upon the structure of the cartilage or bone in a typical arthritic joint. The availability of HA from biotechnologically-derived bacterial fermentation techniques and hydrolysis with hyaluronidase enzymes provides a lower molecular weight fraction, typically of the order of 200,000 Daltons. This HA fraction is advantageously combined with lipids derived from Perna canaliculus, both as an oral and topical application, for the treatment of arthritis and other inflammatory conditions. The use of a combined product produces clinically better results than the use of the individual components alone.

### EXAMPLE 1

### A Double-Blind Placebo Controlled Crossover Clinical Trial Comparing the Efficacy of a Green Lipped Mussel Lipid Extract And Hyaluronic Acid Alone and in Combination on Lameness in Horses

Arthritis is a significant problem in both humans and animals that may occur at any age but is particularly common in older individuals. In horses, both degenerative and inflammatory arthropathies may occur, but the most common form of joint disease is osteoarthritis, a complex, progressive disease characterized by the degeneration of articular cartilage and by the formation of new bone (osteophytes) at joint margins. It is often the result of trauma, low grade or acute, sustained over a working life.

Inflammation of the synovial membrane may also be present in many cases of OA, but is a variable feature throughout the course of the disease. Conversely, synovitis is the major pathological feature of the inflammatory joint diseases, such as rheumatoid arthritis. Structural damage may exist for some time before clinical signs of OA are apparent, and most cases ultimately present with stiffness or lameness. Lameness, attributed to a combination of joint pain and restricted movement of the joint, may be gradual in onset or may present acutely following minor trauma or excessive exercise.

A crossover double blind and randomised study was designed to evaluate the efficacy of GLM lipid extract and HA alone and in combination in the treatment of lameness in horses.

Subjects

This study used mixed breed/sex horses (7-18 y old) that had exhibited varying degrees of arthritic signs, living at an horse sanctuary. Any horse exhibiting arthritic signs for 4 months or less and horses that did not consistently exhibit arthritic signs were excluded from the study.

Study Design

Qualifying horses were initially examined and randomly assigned to receive four capsules of the placebo, or containing 50 mg GLM, or 50 mg Hyaluronate, or a mixture of 50 mg GLM and 50 mg Hyaluronate daily. Randomisation was computer generated in balanced blocks of the four treatment regimes and was crossover in design. No other medications were administered.

Measurements

Evaluations of arthritic/musculoskeletal signs were carried out by a veterinarian and research assistant at wk 0 and every two weeks thereafter until the end of the trial. All parameters were scored on a scale of 1 to 10 according to severity and symptom improvement where 1 was severe disease symptomology and 10 indicated a disease free condition score and the results are shown in Table 1 and FIG. 4.

Each horse was scored for mobility (average of individual scores for lameness in walking, trotting, turning and any other musculoskeletal abnormality). Individual joints (neck, back, carpus, elbow and shoulder or tarsus, stifle and hip) of each limb were individually scored for degree of pain, swelling, crepitus and reduction in range of movement. Horses were also filmed for gait analysis and scored. Summation of the mobility score and all individual joint scores for each horse comprised their total arthritic/musculoskeletal score.

Adverse Reactions

No adverse reactions were observed or reported.

Results

The individual data obtained for the six horses entered into the lameness study and the statistical analysis of the data was evaluated. The clinical assessments scores were assessed as a mean clinical lameness score. Supplementing the horses with GLM lipids reduced (P<0.01) their degree of lameness within 2 weeks of treatment. Hyaluronate appeared to have little influence on it own but when given with GLM lipids there was a greater improvement in locomotory score within 14 days. By the end of the study phase at 28 days, locomotory score was similar between the GLM lipids and the GLM lipids plus Hyaluronate treatment groups. The data suggests that lame horses benefit more quickly if the two compounds are administered together and the mid phase improvement is biologically significant at 14.5% over the GLM Lipids alone.

TABLE 1

| | | Mid Phase 14 | End Phase 28 |
|---|---|---|---|
| | Components | Days | Days |
| A | BioActive lipids | 6.3 ± 0.61^{ab} | 7.6 ± 0.8^{bc} |
| D | Inert Carrier | 1.7 ± 0.77^{ac} | 0.97 ± 0.76^{bd} |
| E | Hyaluronate | 2.6 ± 0.47^{bc} | 2.8 ± 0.48^{ef} |
| F | Bioactive Lipids + Hyaluronate | 7.2 ± 0.75^{cc} | 7.44 ± 0.67^{df} |

Values are presented as Mean ± SEM. Values in columns with the same superscript differ significantly: ^{a}P < 0.05, ^{bcd}P < 0.01, ^{ef}P < 0.001

What is claimed is:

1. A pharmaceutical or veterinary composition comprising a hyaluronic acid or a salt thereof or an ester of hyaluronic acid with an alcohol of the aliphatic, heterocyclic or cycloaliphatic series, or a sulphated form of hyaluronic acid, together with an extract of fatty acids from Perna canaliculus providing eicosatetraenoic acid.

2. A composition as claimed in claim 1, for topical administration.

3. A composition as claimed in claim 2, further comprising a pharmaceutically or veterinarily acceptable diluent or carrier.

4. A composition as claimed in claim 3, formulated as a cream, ointment, lotion, poultice or gel or skin patch.

5. A composition as claimed in claim 1, containing a concentration of hyaluronic acid or a said derivative thereof of from 1 to 20 wt % based on the total weight of the composition.

6. A composition as claimed in claim 1, containing a concentration of said eicosatetraenoic acid of from 1 to 20 wt %.

7. A composition as claimed in claim 1, for oral administration.

8. A composition as claimed in claim 7, further comprising a pharmaceutically or veterinarily acceptable diluent or carrier.

9. A composition as claimed in claim 8, formulated as a syrup, solution, capsule, lozenge suppository, tablet, chewable tablet, rapid dissolving wafer, or gelatin or non-gelatin capsule.

10. A composition as claimed in claim 7, in unit dosage form, wherein each unit dosage form contains from 5 to 500 mg of hyaluronic acid or said derivative thereof.

11. A composition as claimed in claim 7, in unit dosage form, wherein each unit dosage form contains from 5 to 500 mg of said eicosatetraenoic acid.

12. A composition as claimed in any claim 7, in liquid dosage form, wherein composition contains a concentration of from 1 to 20% wt/vol of hyaluronic acid or said derivative thereof.

13. A composition as claimed in claim 7, in liquid dosage form, wherein composition contains a concentration of from 1 to 20% wt/vol of said eicosatetraenoic acid.

14. A composition as claimed in claim 1, wherein the weight ratio of said hyaluronic acid or derivative thereof to said eicosatetraenoic acid is from 1:1 to 1:100.

15. (canceled)

16. (canceled)

17. A composition as claimed in claim 1, wherein eicosatetraenoic acid constitutes at least 0.05 wt % of the fatty acid content of the composition.

18. A composition as claimed in claim 1, wherein said hyaluronic acid or derivative thereof is a low molecular weight form, having a molecular weight of from 50,000 to 500,000.

19. A composition as claimed in claim 18, wherein said hyaluronic acid or derivative thereof is a low molecular weight form, having a molecular weight of from 150,000 to 250,000.

20. A method of therapy comprising administering to a mammal suffering from an arthritic condition or other inflammatory condition or in need of prophylaxis in respect of such a condition an effective amount of a hyaluronic acid or a slat thereof or an ester of hyaluronic acid with an alcohol of the aliphatic, heterocyclic or cycloaliphatic series, or a sulphated form of hyaluronic acid, together with an extract of fatty acids from Perna canaliculus providing eicosatetraenoic acid.
